(19) United States
(12) Patent Application Publication
(10) Pub. No.: US 2023/0025890 A1
(43) Pub. Date: Jan. 26, 2023

(54) Transcatheter Valve To Treat Small Native Mitral Anatomy

(71) Applicant: Tendyne Holdings, Inc., St. Paul, MN (US)

(72) Inventors: Heath Marnach, Minneapolis, MN (US); William Peckels, Robbinsdale, MN (US)

(73) Assignee: Tendyne Holdings, Inc., St. Paul, MN (US)

(21) Appl. No.: 17/826,268

(22) Filed: May 27, 2022

Related U.S. Application Data
(60) Provisional application No. 63/223,594, filed on Jul. 20, 2021.

(51) Int. Cl. A61F 2/24

(52) U.S. Cl. CPC A61F 2/2418; A61F 2220/0008; A61F 2/2427

(57) ABSTRACT

A prosthetic mitral valve with improved blood flow to the left ventricular outflow tract (LVOT). The prosthetic mitral valve includes an expandable outer stent having an atrial end and a ventricular end, and an expandable inner stent attached to and at least partially positioned within the outer stent. The inner stent has an inflow end, an outflow end and a connector securing a tether. A valve assembly including a cuff and a plurality of leaflets may be disposed within the inner stent. The outer stent is expandable from a delivery condition in which the outer stent is axially elongated to a deployed condition in which a first portion of the outer stent is folded upon a second portion of the outer stent to define a flange for engaging an atrial surface of a native valve annulus and to stabilize the prosthetic heart valve within the native valve annulus.

### CROSS-REFERENCE TO RELATED APPLICATIONS

This application claims the benefit of the filing date of U.S. Provisional Patent Application No. 63/223,594 filed Jul. 20, 2021, the disclosure of which is hereby incorporated herein by reference.

### BACKGROUND OF THE DISCLOSURE

The present disclosure relates to expandable prosthetic heart valves, and more particularly, to apparatus and methods for stabilizing an expandable prosthetic heart valve within a native annulus of a patient.

Prosthetic heart valves that are collapsible to a relatively small circumferential size can be delivered into a patient less invasively than valves that are not collapsible. For example, a collapsible and expandable valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible and expandable prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed to reduce its circumferential size.

When a collapsed prosthetic valve has reached the desired implant site in the patient (e.g., at or near the native annulus of the patient's heart valve that is to be repaired by the prosthetic valve), the prosthetic valve can be deployed or released from the delivery apparatus and expanded to its full operating size. For balloon-expandable valves, this generally involves releasing the entire valve, assuring its proper location, and then expanding a balloon positioned within the valve stent. For self-expanding valves, on the other hand, the stent automatically expands as the stent is withdrawn from the delivery apparatus.

The clinical success of collapsible and expandable heart valves is dependent, in part, on the anchoring of the valve within the native valve annulus. Self-expanding valves typically rely on the radial force exerted by expanding the stent against the native valve annulus to anchor the prosthetic heart valve. However, if the radial force is too high, the heart tissue may be damaged. If, instead, the radial force is too low, the heart valve may move from its deployed position and/or migrate from the native valve annulus, for example, into the left ventricle.

Movement of the prosthetic heart valve may result in the leakage of blood between the prosthetic heart valve and the native valve annulus. This phenomenon is commonly referred to as paravalvular leakage (PVL). In mitral valves, paravalvular leakage enables blood to flow from the left ventricle back into the left atrium during ventricular systole, resulting in reduced cardiac efficiency and strain on the heart muscle.

Anchoring prosthetic heart valves within the native valve annulus of a patient, especially within the native mitral valve annulus, can be difficult. The native mitral valve annulus, for instance, has reduced calcification or plaque compared to the native aortic valve annulus which can make for a less stable surface to anchor the prosthetic heart valve. For this reason, collapsible and expandable prosthetic mitral valves often include additional anchoring features such as barbs that engage underneath the annulus and/or coils that capture native leaflets, or that wrap around chordae tendineae, thereby stabilizing the prosthetic heart valve within the native annulus.

Despite the improvements that have been made to anchoring collapsible and expandable prosthetic heart valves, shortcomings remain. For example, to accommodate the additional anchoring features, prosthetic heart valves often extend at least partially into the ventricle, which can impede blood flow to the Left Ventricular Outflow Tract (LVOT). The challenges of anchoring a prosthetic heart valve within a native mitral valve annulus of a patient, without impeding blood flow to the LVOT, is only exacerbated when a patient has a small native mitral anatomy.

### BRIEF SUMMARY OF THE DISCLOSURE

In accordance with a first aspect of the present disclosure, a collapsible and expandable prosthetic heart valve having a low-ventricular profile is provided. Among other advantages, the prosthetic heart valve is designed to be securely anchored (e.g., tethered) within the native mitral valve annulus without projecting into the ventricle. As a result, the prosthetic heart valve disclosed herein minimizes the obstruction of blood flow to the LVOT.

One embodiment of the prosthetic heart valve includes a prosthetic heart valve having an expandable inner stent with an inflow end and an outflow end, a valve assembly disposed within the stent including a cuff and a plurality of leaflets, an outer stent secured to and at least partially surrounding the inner stent, and a tether. The outer stent defines an atrial end and a ventricular end and is expandable from a delivery condition in which the outer stent is axially elongated to a deployed condition in which a first portion of the outer stent is folded upon a second portion of the outer stent such that the first and second portions collectively form a flange sized to engage an atrial surface of a native valve annulus.

In another embodiment, the prosthetic heart valve includes an expandable inner stent defining an inflow end and an outflow end, a valve assembly disposed within the inner stent including a cuff and a plurality of leaflets, an outer stent secured to and at least partially surrounding the inner stent, and a tether. The outer stent defines a first foldable portion, a second foldable portion, a body portion, a first junction between the second foldable portion and body portion, and a second junction between the first foldable portion and the second foldable portion. The outer stent is expandable from a delivery condition in which the first foldable portion, the second foldable portion and the body portion are substantially aligned to a deployed condition in which the second foldable portion pivots outwardly about the first junction relative to the body portion and the first foldable portion curls about the second junction such that the first foldable portion and the second foldable portion collectively form a double walled flange sized to engage an atrial surface of a native valve annulus. A sealing cuff is disposed on a surface of the double walled flange to seal a space between the prosthetic heart valve and the native mitral valve annulus.

A method of implanting a prosthetic heart valve within a native heart valve annulus is provided herein and includes delivering a delivery device to a target site adjacent to a native valve annulus while the delivery device holds a prosthetic heart valve including an inner stent, a valve assembly disposed within the stent, an outer stent secured to and at least partially surrounding the inner stent and a tether; deploying the prosthetic heart valve from the delivery device and allowing a first portion of the outer stent to fold onto a second portion of the outer stent to define a flange; engaging the flange against an atrial surface of a native annulus; tensioning the tether; and securing the tether to the wall of the heart.

### BRIEF DESCRIPTION OF THE DRAWINGS

Various embodiments of the present disclosure are described herein with reference to the drawings, wherein:

FIG. 1 is a highly schematic cutaway view of the human heart, showing two approaches for delivering a prosthetic mitral valve to an implantation location;

FIG. 2 is a highly schematic representation of a native mitral valve and associated cardiac structures;

FIG. 3 is a highly schematic longitudinal cross-section of a prosthetic mitral valve according to an embodiment of the present disclosure;

FIG. 4 is a side elevational view of an inner stent of the prosthetic mitral valve of FIG. 3;

FIG. 5 is a side elevational view of an outer stent of the prosthetic mitral valve of FIG. 3;

FIG. 6 is a highly schematic cutaway view of the human heart, showing the prosthetic mitral valve of FIG. 3 implanted within the native mitral valve annulus.

FIGS. 7A-7D are highly schematic partial longitudinal cross-sections showing deployment of the prosthetic mitral valve of FIG. 3 from a delivery device for implantation within a native annulus; and

FIG. 8 is a highly schematic view of the prosthetic mitral valve of FIG. 3 implanted within the native mitral valve annulus.

### DETAILED DESCRIPTION

Blood flows through the mitral valve from the left atrium to the left ventricle. As used herein in connection with a prosthetic heart valve, the term “inflow end” refers to the end of the heart valve through which blood enters when the valve is functioning as intended, and the term “outflow end” refers to the end of the heart valve through which blood exits when the valve is functioning as intended. Also as used herein, the terms “substantially,” “generally,” “approximately,” and “about” are intended to mean that slight deviations from absolute are included within the scope of the term so modified.

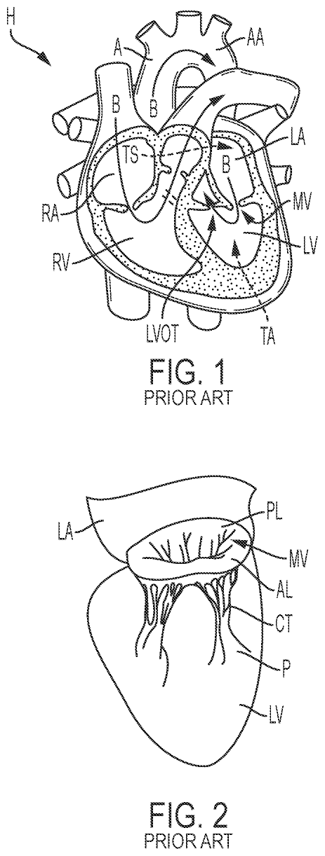

FIG. 1 is a schematic cutaway representation of a human heart H. The human heart includes two atria and two ventricles: right atrium RA and left atrium LA, and right ventricle RV and left ventricle LV. Heart H further includes aorta A, aortic arch AA and left ventricular outflow tract LVOT. Disposed between left atrium LA and left ventricle LV is mitral valve MV. The mitral valve, also known as the bicuspid valve or left atrioventricular valve, is a dual-flap that opens as a result of increased pressure in left atrium LA as it fills with blood. As atrial pressure increases above that in left ventricle LV, mitral valve MV opens and blood flows into the left ventricle. When left ventricle LV contracts during systole, blood is pushed from the left ventricle, through left ventricular outflow tract LVOT and into aorta A. Blood flows through heart H in the direction shown by arrows “B”.

A dashed arrow, labeled “TA”, indicates a transapical approach of implanting a prosthetic heart valve, in this case to replace the mitral valve. In the transapical approach, a small incision is made between the ribs of the patient and into the apex of left ventricle LV to deliver the prosthetic heart valve to the target site. A second dashed arrow, labeled “TS”, indicates a transseptal approach of implanting a prosthetic heart valve in which the delivery device is inserted into the femoral vein, passed through the iliac vein and the inferior vena cava into right atrium RA, and then through the atrial septum into left atrium LA for deployment of the valve. Other approaches for implanting a prosthetic heart valve are also possible and may be used to implant the collapsible prosthetic heart valve described in the present disclosure.

FIG. 2 is a more detailed schematic representation of native mitral valve MV and its associated structures. As previously noted, mitral valve MV includes two flaps or leaflets, posterior leaflet PL and anterior leaflet AL, disposed between left atrium LA and left ventricle LV. Cord-like tendons, known as chordae-tendineae CT, connect the two leaflets to the medial and lateral papillary muscles P. During atrial systole, blood flows from higher pressure in left atrium LA to lower pressure in left ventricle LV. When left ventricle LV contracts during ventricular systole, the increased blood pressure in the chamber pushes the posterior and anterior leaflets to close, preventing the backflow of blood into left atrium LA. Since the blood pressure in left atrium LA is much lower than that in left ventricle LV, the leaflets attempt to evert to low pressure regions. Chordae tendineae CT prevent the eversion by becoming tense, thus pulling on the leaflets and holding them in the closed position.

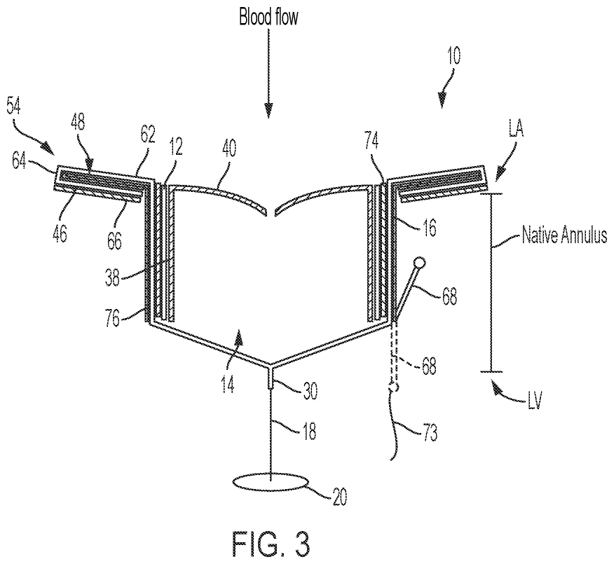

FIG. 3 is highly schematic longitudinal cross-section of a collapsible and expandable prosthetic heart valve 10 according to an embodiment of the present disclosure. For balloon-expandable variants, prosthetic heart valve 10 may be expandable, but not collapsible, or not readily collapsible, once expanded. When used to replace native mitral valve MV (shown in FIGS. 1 and 2), prosthetic valve 10 may have a low profile so as minimize any interference with the heart's electrical conduction system pathways, atrial function or blood flow to the left ventricular outflow tract LVOT (shown in FIG. 1).

Prosthetic heart valve 10 includes an inner stent 12 securing a valve assembly 14, an outer stent 16 attached to and disposed around the inner stent, and a tether 18 configured to be secured to an apical pad 20. Both the inner stent 12 and the outer stent 16 may be formed from biocompatible materials that are capable of self-expansion, for example, shape-memory alloys such as nitinol. Alternatively, inner stent 12 and/or outer stent 16 may be balloon expandable or expandable by another force exerted radially outward on the stent. When expanded, outer stent 16 folds upon itself to form a flange that engages an atrial surface of the native valve annulus and assists in anchoring inner stent 12 and valve assembly 14 within the native valve annulus when tether 18 is tensioned.

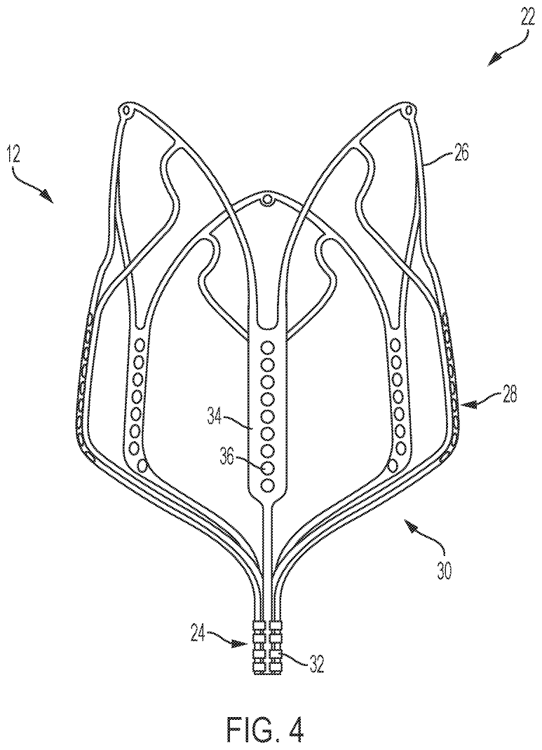

Referring to FIG. 4, inner stent 12 extends along a longitudinal axis between an inflow end 22 and an outflow end 24. In one example, inner stent 12 is formed by laser cutting a predetermined pattern into a metallic tube, such as a nitinol tube, to form four portions: cusps 26, a post portion 28, a strut portion 30 and a valve stem 32 (or “tether clamp”) that secures tether 18 (shown in FIG. 3). Strut portion 30 may include, for example, six struts that extend radially inward from post portion 28 to tether clamp 32. When inner stent 12 is expanded, strut portion 30 forms a radial transition between post portion 28 and tether clamp 32 that facilitates crimping of the inner stent when tether 18 is retracted within a delivery device. Post portion 28 may also include six longitudinal posts 34 having a plurality of bores 36 for securing valve assembly 14 to the inner stent 12 by one or more sutures and for securing the outer stent 16 to the inner stent by one or more sutures. As shown in FIG. 4, three cusps 26 are positioned at the inflow end 22 of inner stent 12. Each cusp 26 is circumferentially disposed between a pair of non-adjacent longitudinal posts 34 with a single longitudinal post positioned between each of the non-adjacent longitudinal posts.

With additional reference to FIG. 3, valve assembly 14 may be secured to inner stent 12 by suturing the valve assembly to longitudinal posts 34. Valve assembly 14 includes a cuff 38 and a plurality of leaflets 40 that open and close collectively to function as a one-way valve. Cuff 38 and leaflets 40 may be wholly or partly formed of any suitable biological material, such as bovine or porcine pericardium, or biocompatible polymer, such as polytetrafluorethylene (PTFE), urethanes and the like. The bores 36 of longitudinal posts 34 facilitate the suturing (or connection via another fastener or attachment mechanism) of the leaflet commissure to the post portion 28 of inner stent 12.

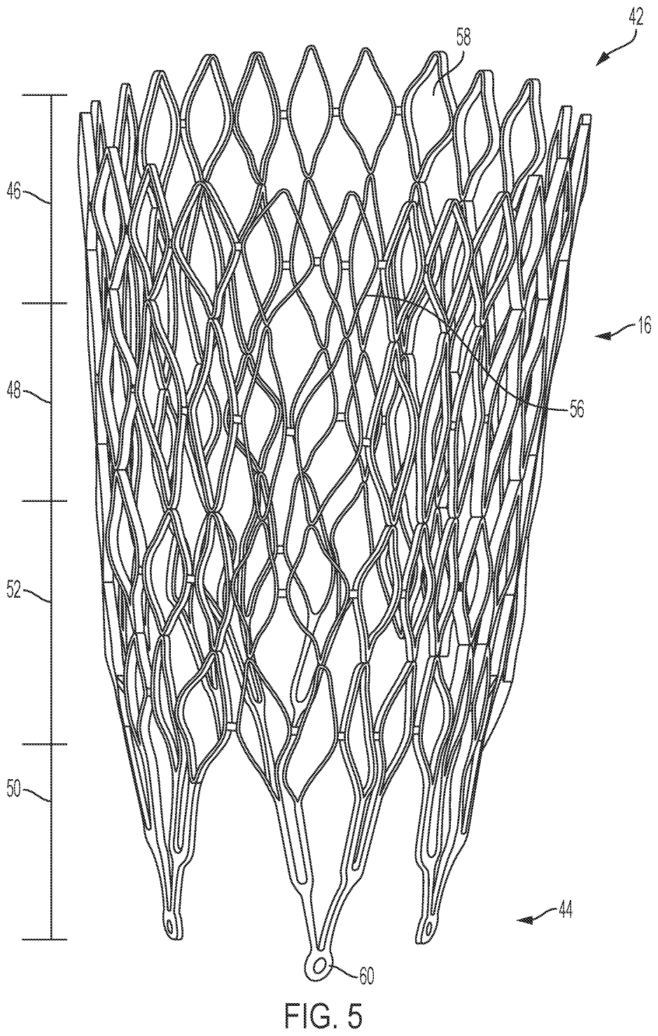

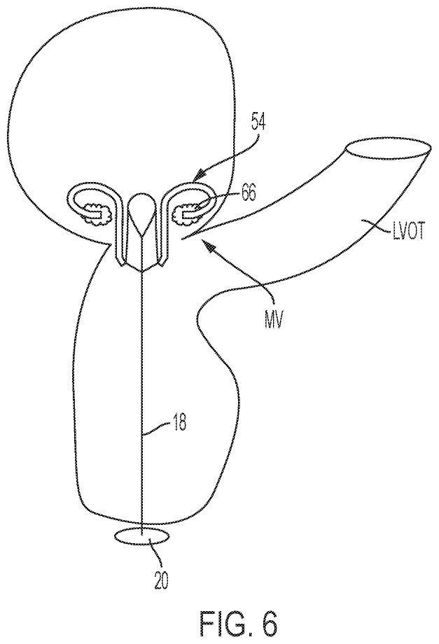

Referring now to FIGS. 3 and 5, outer stent 16 extends between an atrial end 42 and a ventricular end 44 along the same longitudinal axis as inner stent 12. In one example, outer stent 16 is formed by laser cutting a predetermined pattern into a metallic tube, such as a self-expanding nitinol tube, to define four portions: a first foldable portion 46 adjacent the atrial end; a second foldable portion 48 adjacent the first foldable portion; attachment features 50 adjacent the ventricular end; and a body portion 52 disposed between the second foldable portion and the attachment features. Outer stent 16 is shown in FIG. 5 in an expanded, yet axially elongated (e.g., not folded) condition, in order to clearly illustrate the structure of the first and second foldable portions. It will be understood, however, that outer stent 16 is heat-set (or otherwise pre-set) in a manner that, when expanded, causes the first foldable portion 46 to curl upon the second foldable portion 48 such that the first and second foldable portions collectively define a flange 54 as shown in FIGS. 3 and 6.

The first foldable portion 46, the second foldable portion 48 and the body portion 52 of outer stent 16 may include a plurality of struts 56 that form cells 58 extending about the outer stent in one or more annular rows. Cells 58 may be substantially the same size around the perimeter of stent 16 and along the length of the stent. Alternatively, cells 58 within the body portion 52 and closer to the atrial end 42 of outer stent 16 may be larger than the cells within the body portion near the ventricular end 44 of the stent. The attachment features 50 may extend from the struts 56 forming apices of adjacent cells 58 that lie within the ventricular-most row of cells of outer stent 16. Attachment features 50 may define an eyelet 60 that facilitates the suturing (or connection via another fastener or attachment mechanism) of outer stent 16 to the longitudinal posts 34 of inner stent 12, thereby securing the inner and outer stents together. In one example, attachment features 50 may be sutured to a single bore 36 of a longitudinal post 34, proximate to the outflow end 24 of inner stent 12.

With additional reference to FIGS. 3, 6 and 7A-7D, when outer stent 16 is expanded, the second foldable portion 48 of the outer stent may bend outwardly, approximately 90° (e.g., in a direction generally orthogonal to the longitudinal axis) about a first junction 62, formed between the second foldable portion and the body portion 52 of the outer stent. Expansion of outer stent 16 may also cause the first foldable portion 46 to curl approximately 180° about a second junction 64, formed between the first foldable portion and the second foldable portion, such that the first foldable portion substantially overlaps the second foldable portion and the combination of the first and second foldable portions collectively form a radially extending flange 54 having a “double wall.”

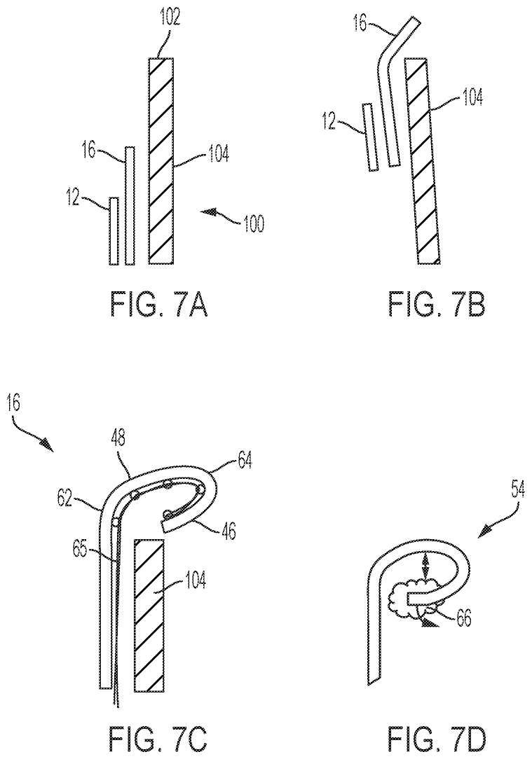

As shown in FIG. 3, the first foldable portion 46 of outer stent 16 may curl underneath the second foldable portion 48 of the stent. In other words, a surface of first foldable portion 46, defining a luminal surface of the outer stent 16 when the outer stent is in a delivery condition, may curl to engage the atrial surface of the native mitral valve annulus when the stent is expanded to the deployed condition. Alternatively, outer stent 16 may be heat-set such that first foldable portion 46 curls in an opposite direction and lies on top of the second foldable portion 48 when the outer stent is transitioned to the deployed condition. In this manner, when outer stent 16 is in the deployed condition, an abluminal surface of the second portion 48 of the outer stent may engage the atrial surface of the native mitral valve annulus. A series of loops, as shown in FIG. 7C may extend in a longitudinal direction along an abluminal surface of the first foldable portion 46 and the second foldable portion 48 of outer stent 16. A suture 65 may be threaded through each of the loops beginning from a loop nearest the ventricular end 44 of outer stent 16, around a loop adjacent to the atrial end 42 of the outer stent, and back through each of the loops toward ventricular end of the stent. The two terminal ends of suture 65 can thus be manipulated (e.g., pulled in a proximal direction) by a user to assist in curling the first foldable portion 46 of outer stent 16 relative to the second foldable potions 48 of the outer stent.

In a preferred embodiment, a sealing cuff 66 is disposed on a surface of flange 54 that engages an atrial surface of the native mitral valve annulus when prosthetic heart valve 10 is implanted within the native mitral valve. Sealing cuff 66 may be formed of a fabric, or a biologic or synthetic tissue, to seal the space between prosthetic heart valve 10 and the native mitral valve annulus. In one example, the material of sealing cuff 66 may be segmented into a plurality of discrete pieces, each of which is sutured or otherwise secured to struts 56 forming a single cell 58 or, alternatively, to the struts forming a perimeter around a relatively few number of cells. In this regard, each of the discrete pieces can flex relative to one another so as to not inhibit the bending of first foldable portion 46 and second foldable portion 48 relative to body portion 52. Alternatively, sealing cuff 66 may be formed of a single piece of material if the material is stretchable or otherwise does not inhibit outer stent 16 from transitioning from the delivery condition to the deployed condition and the formation of flange 54.

With specific reference to FIG. 3, flange 54 is designed to protrude into the left atrium LA and engage an atrial surface of the native mitral valve annulus when the body portion 52 of outer stent 16 is disposed within the native mitral valve MV, thereby preventing the prosthetic heart valve 10 from migrating into left ventricle LV. Prosthetic heart valve 10 is anchored within the native mitral valve annulus by the radial force exerted by the body portion 52 of outer stent 16 against the native annulus, the flange 54 engaging the atrial surface of the native valve annulus and tether 18 anchored to the ventricular wall of the heart. The flange is the only portion of prosthetic heart valve 10 that is designed to protrude out from the native mitral valve annulus. Put another way, when expanded, inner stent 12 and outer stent 16 are designed with a low-ventricular profile (e.g., a height between about 5 mm and about 15 mm) so as to not extend into left ventricle LV. In this manner, prosthetic heart valve 10 does not interfere with blood flow to the left ventricular outflow tract LVOT. The low-ventricular profile design is possible, in part, because of the relatively rigid double walled flange 54. When prosthetic heart valve 10 is implanted within the native mitral valve annulus, tether 18 may be tensioned with a force that is greater than would be permitted with a similarly constructed prosthetic heart valve having a single walled flange and, as a result of the increased tension, the need for additional anchoring features is alleviated. Put differently, applying the same tension on a tether of a similarly constructed prosthetic heart valve having a single walled flange may cause the single walled flange to bend and result in the heart valve being pulled through the native mitral valve annulus into the left ventricle. On the other hand, if the physician under tensions the prosthetic heart valve having a single walled flange in trepidation of pulling the prosthetic heart valve through the native mitral valve annulus and into the left ventricle, then the patient is at risk of developing PVL.

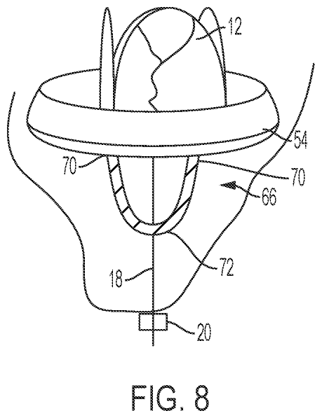

Systolic Anterior Motion (SAM) prevention features may optionally be provided, for example, on outer stent 16. SAM (e.g., the displacement of the free edge of native anterior leaflet AL toward left ventricular outflow tract LVOT) can result in severe left ventricular outflow tract LVOT obstruction and/or mitral regurgitation. To prevent the occurrence of SAM, or at least significantly reduce its likelihood, a pivot arm 68 (shown in FIGS. 3 and 8) may be attached to an anterior side of outer stent 16. As illustrated in FIG. 8, pivot arm 68 may include two arm segments that are attached at ends 70 to the struts 56 of outer stent 16 at an attachment point, with a looped portion 72 connecting the other ends of the arm segments together. Pivot arm 68 may be pivotally mounted to outer stent 16 so as to be transitionable from a collapsed condition in which the looped portion 72 faces in a ventricular direction (e.g., away from the inflow end 22 of inner stent 12) during delivery of prosthetic heart valve 10 into the patient, to an expanded condition in which the arm segments pivot about an attachment point such that the looped portion faces substantially in an atrial direction (e.g., toward the inflow end of inner stent) and clamps the native anterior leaflet between the pivot arm and an abluminal surface of the outer stent. A suture 73, or other cord, may be looped through a ring provided on pivot arm 68 and used to prevent the pivot arm from transitioning from the collapsed condition to the expanded condition during delivery until after the physician releases tension on the cord.

In a preferred embodiment, as shown in FIG. 3, prosthetic heart valve 10 may include an inner skirt 74 and an outer skirt 76. Outer skirt 76 may be disposed about the abluminal surface of the body portion 52 of outer stent 16 and may be formed of a fabric, such as polyester, that promotes tissue ingrowth. The fabric of outer skirt 76 is preferably an independent and discrete material from the material forming sealing cuff 66. In this regard, outer skirt 76 will not inhibit the transitioning of outer stent 16 from the delivery condition to the deployed condition and the formation of flange 54. In other embodiments, however, the fabric of outer skirt 76 may be integrally formed with sealing cuff 66, or otherwise connected to the sealing cuff, if the material is formed of a stretchable material that will not interfere with the formation of flange 54. Inner skirt 74 may be disposed about the luminal surface of outer stent 16 and may be formed of any suitable biological material, such as bovine or porcine pericardium, or any suitable biocompatible polymer, such as PTFE, urethanes or similar materials. The biological tissue may bridge inner stent 12 and outer stent 16 and reinforced by sutures to prevent back pressure and the ballooning of the material. When prosthetic heart valve 10 is implanted within the native mitral valve annulus, inner skirt 74 and outer skirt 76 act in combination with sealing cuff 66 to prevent mitral regurgitation, or the flow of blood between the prosthetic heart valve 10 and the native mitral valve annulus. In one embodiment, inner skirt 74 and outer skirt 76 extend only between the atrial end 42 of outer stent 16 and the junction between the body portion 52 and the attachment features 50 of the outer stent to facilitate the suturing of the outer stent to inner stent 12.

Use of prosthetic heart valve 10 to repair a malfunctioning native heart valve, such as a native mitral valve, or a previously implanted and malfunctioning prosthetic heart valve, will now be described with reference to FIGS. 3, 6, 7A-7D and 8. Although prosthetic heart valve 10 is described herein as repairing a native mitral valve using a transapical approach, it will be appreciated that the prosthetic heart valve may be used to repair the native mitral valve using a transseptal or other suitable approach, as well as to repair other cardiac valves, such as the aortic valve, using any suitable approach.

With a first end of tether 18 secured to the clamp 32 of inner stent 12, a physician may pull the free end of the tether through a loading device (not shown), such as a funnel, to crimp or collapse inner stent 12 and transition outer stent 16 from the expanded or deployed condition to the collapsed or delivery condition. After prosthetic heart valve 10 has been collapsed, the prosthetic heart valve may be loaded within a delivery device 100 with the free end of tether 18 extending back towards the trailing end (not shown) of the delivery device such that it can be manipulated by a physician.

After an incision has been made between the ribs of the patient and into the apex of the heart, delivery device 100 may be introduced into the patient using a transapical approach and delivered to an implant site adjacent the native mitral valve annulus. Once delivery device 100 has reached the target site, with a leading end 102 of delivery sheath 104 disposed within left atrium LA, the delivery sheath may be retracted to expose the atrial end 42 of outer stent 16, thereby allowing outer stent 16 to expand and transition from the delivery condition to the deployed condition.

As shown in FIGS. 7A-7D, expansion of outer stent 16 will cause the second foldable portion 48 of the stent to pivot radially outward about first junction 62 until the second foldable portion is oriented in a direction generally orthogonal to the longitudinal axis. Expansion of outer stent 16 will also cause the first foldable portion 46 to curl about second junction 64 until the first foldable portion lies underneath the second foldable portion, thereby forming double walled flange 54. In certain embodiments, the curling of the first foldable portion 46 about the second junction 64 may be assisted by the physician pulling the terminal end of suture 65 in a proximal direction. After the double walled flange 54 has been formed the physician may then retract delivery device 100 in a proximal direction until flange 54 and, more specifically, first foldable portion 46 is engaged against the atrial surface of the native mitral annulus. A pusher or similar member may be utilized to prevent the prosthetic heart valve 10 from retracting as the delivery device 100 is retracted. With flange 54 engaged against the atrial surface of the mitral valve annulus, the physician may further unsheathe prosthetic heart valve 10, allowing the body portion 52 of outer stent 16 to expand and engage the native valve annulus, while also allowing inner stent 12 to expand from the collapsed condition to the expanded condition within the outer stent. After the inner stent 12 and the outer stent 16 have been expanded, a physician may determine whether prosthetic heart valve 10 has restored proper blood flow through the native mitral valve. More particularly, the physician may determine: 1) whether valve assembly 14 is functioning properly; and 2) whether the prosthetic heart valve 10 has been properly seated within the native valve annulus to form a seal between the prosthetic heart valve and the native mitral valve annulus.

In the event that the physician determines that the valve assembly 14 is malfunctioning or that prosthetic heart valve 10 is positioned incorrectly within the native mitral annulus, the physician may recapture the prosthetic heart valve. To recapture prosthetic heart valve 10, the physician may pull tether 18 toward the trailing end of delivery device 100 thereby retracting the prosthetic heart valve and engaging the strut portion 30 of inner stent 12 against the leading end 102 of delivery sheathe 104 to crimp the inner stent, and with it outer stent 16, to a diameter capable of being inserted into the leading end the delivery sheathe. If valve assembly 14 was working as intended, but prosthetic heart valve 10 was mispositioned within the native mitral valve annulus, the physician may only need to partially collapse the prosthetic heart valve within delivery device 100 before repositioning the delivery sheathe with respect to the native mitral annulus and redeploying the prosthetic heart valve as previously described. Alternatively, if valve assembly 14 was malfunctioning, prosthetic heart valve 10 may be completely recaptured and removed from the patient. The physician may then repeat the procedure described above with a different prosthetic heart valve 10.

In some instances, the physician may find it desirable to secure the native anterior leaflet AL of native mitral valve MV to the outer stent 16 of prosthetic mitral valve 10 to prevent SAM. When desired, the physician may use prosthetic heart valve 10 having a pivot arm 68, which when unsheathed and when tension is released from suture 73, will automatically pivot from the collapsed condition to an expanded condition to secure the native leaflet to prosthetic heart valve 10 and away from the left ventricular outflow tract.

After the physician has confirmed that prosthetic heart valve 10 has been properly positioned, and leaflets 40 are properly coapting, the physician may insert apical pad 20 through the incision before coupling tether 18 and apical pad 20 and tensioning the tether. As shown in FIG. 7D, tensioning the tether by pulling the free end of tether 18 towards the trailing end of delivery sheath 104 causes the first foldable portion 46 and the second foldable portion 48 to compress together and also causes the sealing cuff 66 disposed on flange 54 to press against the atrial surface of the native mitral valve annulus, thereby sealing the space between the flange and the atrial surface of the native mitral valve annulus. The double walled flange 54 of prosthetic heart valve 10 thus stabilizes the prosthetic heart valve within the native mitral valve annulus and prevents paravalvular leakage, while allowing the low profile prosthetic heart valve to sit relatively high within the native mitral annulus (e.g., within a plane arranged at an atrial surface of the annulus) such that the prosthetic heart valve does not impede blood flow to the left ventricular outflow tract.

Apical pad 20, which may be positioned in contact with an exterior surface of left ventricle LV at the transapical puncture site, may then be locked to tether 18, preventing the tether from releasing the tension. The physician may then cut the tether located outside of the heart before removing the cut portion of the tether and delivery device 100 from the patient. With prosthetic heart valve 10 properly positioned and anchored within the native mitral valve annulus of a patient, the prosthetic heart valve may work as a one-way valve to restore proper function of the heart valve by allowing blood to flow in one direction (e.g., from the left atrium to the left ventricle) while preventing blood from flowing in the opposite direction.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve, comprising:
an expandable inner stent defining an inflow end and an outflow end;
a valve assembly disposed within the inner stent, the valve assembly including a cuff and a plurality of leaflets;
an outer stent secured to and at least partially surrounding the inner stent, the outer stent defining an atrial end and a ventricular end, and being expandable from a delivery condition in which the outer stent is axially elongated to a deployed condition in which a first portion of the outer stent is folded upon a second portion of the outer stent such that the first and second portions collectively form a flange sized to engage an atrial surface of a native valve annulus; and
a tether to secure the prosthetic heart valve within the native valve annulus.

2. The prosthetic heart valve of claim 1, wherein the outer stent further comprises a body portion disposed between the second portion and the ventricular end of the outer stent, the body portion shaped to sit within the native valve annulus when the outer stent is in the deployed condition.

3. The prosthetic heart valve of claim 2, wherein the outer stent defines a first junction between the body portion and the second portion, the second portion being pivotable outwardly about the first junction when the outer stent transitions from the delivery condition to the deployed condition.

4. The prosthetic heart valve of claim 3, wherein the outer stent defines a second junction between the first portion and the second portion, the first portion being bendable underneath the second portion when the outer stent transitions from the delivery condition to the deployed condition.

5. The prosthetic heart valve of claim 3, wherein the outer stent defines a second junction between the first portion and the second portion, the first portion being bendable to lie above the second portion when the outer stent transitions from the delivery condition to the deployed condition.

6. The prosthetic heart valve of claim 3, wherein a distance between the first junction and the ventricular end of the outer stent is between about 5 mm and about 15 mm when the outer stent is in the deployed condition.

7. The prosthetic heart valve of claim 1, further comprising a sealing cuff disposed on a surface of the flange to seal a space between the prosthetic heart valve and the native valve annulus.

8. The prosthetic heart valve of claim 7, wherein the sealing cuff is formed of a stretchable fabric.

9. The prosthetic heart valve of claim 7, wherein the sealing cuff is formed of a plurality of discrete fabric pieces.

10. The prosthetic heart valve of claim 1, further comprising a pivot arm coupled to an anterior side of the outer stent at a pivot point, the pivot arm being pivotable between a collapsed condition in which a free end of the pivot arm extends in a ventricular direction when the outer stent is in the delivery condition and an expanded condition in which a native anterior leaflet is clamped between the pivot arm and the outer stent.

11. The prosthetic heart valve of claim 1, further comprising an apical pad for securing the tether to a ventricular wall of the heart.

12. A prosthetic heart valve, comprising:
an expandable inner stent defining an inflow end and an outflow end;
a valve assembly disposed within the inner stent, the valve assembly including a cuff and a plurality of leaflets;
an outer stent secured to and at least partially surrounding the inner stent, the outer stent defining a first foldable portion, a second foldable portion, a body portion, a first junction between the second foldable portion and the body portion, and a second junction between the first foldable portion and the second foldable portion, the outer stent being expandable from a delivery condition in which the first foldable portion, the second foldable portion and the body portion are substantially aligned to a deployed condition in which the second foldable portion pivots outwardly about the first junction relative to the body portion and the first foldable portion curls about the second junction such that the first foldable portion and the second foldable portion collectively form a double walled flange sized to engage an atrial surface of a native valve annulus;
a sealing cuff disposed on a surface of the double walled flange; and
a tether to anchor the prosthetic heart valve within the native valve annulus.

13. The prosthetic heart valve of claim 12, wherein the first foldable portion curls underneath the second foldable portion when the outer stent is in the deployed condition and the sealing cuff is disposed on a surface of the first foldable portion.

14. The prosthetic heart valve of claim 12, wherein the first foldable portion curls to overlie the second foldable when the outer stent is in the deployed condition and the sealing cuff is disposed on a surface of the second foldable portion.

15. The prosthetic heart valve of claim 12, further comprising a fabric configured to promote ingrowth disposed on an abluminal surface of the body portion.

16. A method of implanting a prosthetic heart valve, comprising:
delivering a delivery device to a target site adjacent to a native valve annulus, the delivery device holding a prosthetic heart valve including an inner stent, a valve assembly disposed within the inner stent, an outer stent secured to and at least partially surrounding the inner stent and a tether;
deploying the prosthetic heart valve from the delivery device and allowing a first portion of the outer stent to curl upon a second portion of the outer stent, the first and second portions collectively forming a flange;
engaging the flange against an atrial surface of a native valve annulus;
tensioning the tether; and
securing the tether to the wall of the heart.

17. The method of claim 16, wherein the tensioning step compresses the first and second portions toward one another.

18. The method of claim 16, wherein the delivering step comprises percutaneously delivering the prosthetic heart valve to the native mitral annulus using a transapical approach.

19. The method of claim 16, wherein the delivering step comprises percutaneously delivering the prosthetic heart valve to the native mitral annulus using a transseptal approach.

20. The method of claim 16, further comprising:
pivoting a pivot arm coupled to an anterior side of the outer stent; and
clamping a native anterior leaflet against an abluminal surface of the outer stent away from a left ventricular outflow tract of a patient.
